(19) United States
(12) Patent Application Publication
(10) Pub. No.: US 2003/0233035 A1
(43) Pub. Date: Dec. 18, 2003

(54) Methods and devices for diagnosing and treating choroid plexus failure

(75) Inventor: Edward Rubenstein, Hillsborough, CA (US)

Correspondence Address: SWANSON & BRATSCHUN L.L.C., 1745 SHEA CENTER DRIVE, SUITE 330, HIGHLANDS RANCH, CO, 80129, US

(73) Assignee: SurroMed, Inc.

(21) Appl. No.: 10/461,154

(22) Filed: Jun. 12, 2003

Related U.S. Application Data
(60) Provisional application No. 60/388,329, filed on Jun. 12, 2002.
(60) Provisional application No. 60/390,866, filed on Jun. 20, 2002.

(51) Int. Cl. A61B 5/00

(52) U.S. Cl. 600/309; 604/65

(57) ABSTRACT

Choroid plexus failure in a subject is diagnosed and treated by measuring the level of one or more components of cerebrospinal fluid, comparing the level to a desired level, and adjusting the level toward the desired level. Treatment can occur using an implantable device.

### CROSS-REFERENCE TO RELATED APPLICATIONS

This application claims the benefit of U.S. Provisional Application No. 60/388,329, “Methods and Apparatus for Diagnosing and Treating Disorders of the Central Nervous System and its Appendages,” filed Jun. 12, 2002; and U.S. Provisional Application No. 60/390,866, “Methods and Apparatus for Diagnosing and Treating Disorders of the Central Nervous System and its Appendages,” filed Jun. 20, 2002, both of which are incorporated herein by reference.

### FIELD OF THE INVENTION

The present invention relates generally to diagnosis and treatment of disorders of the central nervous system. More particularly, it relates to disorders caused by failure of the choroid plexus, a central nervous system tissue that produces cerebrospinal fluid (CSF) and its constituents and transports components between the blood and CSF.

### BACKGROUND OF THE INVENTION

Most water-soluble substances in the blood are prevented from entering brain tissue by one of three structures: the cerebral capillaries, which consist of endothelial cells joined by intercellular tight junctions (the blood-brain barrier); the arachnoid membrane, which surrounds the brain surface and defines a sub-arachnoid space; and the choroid plexus. The arachnoid membrane and the choroid plexus serve as a barrier between the blood and the cerebrospinal fluid (CSF) surrounding the brain. The arachnoid membrane is generally impermeable to water-soluble molecules, whereas the choroid plexus regulates concentrations of molecules in the CSF by active transport. The CSF, which exchanges freely with the interstitial brain fluid, contains nutrients, ions, neuroregulatory substances, and other essential molecules and also buoys the brain and dampens accelerations.

The choroid plexus consists of highly vascularized reddish tufts of tissue distributed in four locations of the brain: the fourth ventricle near the base of the brain, the right and left lateral ventricles, and the centrally located third ventricle. The human choroid plexus has a mass of approximately 2-3 grams. Structurally, the choroid plexus consists of capillaries and other small blood vessels surrounded by a single layer of epithelial cells, one side of which contacts the blood plasma, which filters through the leaky walls of the choroid plexus capillaries, and the other side of which extends into the ventricular CSF. These choroidal epithelial cells are sealed together by tight junctions, which prevent passage of even small water-soluble molecules between the blood and CSF. Certain substances, however, are transported actively across the choroid plexus epithelial cells.

The choroid plexus manufactures about 90% of the CSF (the remaining 10% is derived from the interstitial fluid), in addition to many essential CSF constituents. It also actively transfers molecules from the blood to the CSF (with or without modification) and removes metabolites and other noxious substances from the CSF, both by active transport into the blood and by replacement of old CSF with new CSF. Secretion of new CSF by the choroid plexus induces a pressure gradient that causes CSF to flow out of the ventricular system. The total CSF volume in an adult human, approximately 150-175 ml, is replaced three or four times per day, and a large blood supply to the choroid plexus is necessary to sustain such a high output.

Age-related changes in the choroid plexus are known to occur. For example, psammoma bodies, spherical whorls of collagen surrounding an iron-containing core, tend to accumulate over time. Choroid plexus tissue also exhibits an age-dependent calcification, which occurs in over 80% of patients in their 70's. Also evident in the choroid plexuses of elderly patients are cysts, lipid deposits, fibrosis, and flattening and attenuation of the epithelial layer. Additionally, it has been shown that subjects with a mean age of 77 years secrete about half as much CSF as do 29-year olds (C. May et al., “Cerebrospinal fluid production is reduced in healthy aging,” Neurology 40: 500-503, 1990), and also that elderly patients with dementia secrete about half as much CSF as do elderly patients without dementia (G. D. Silverberg et al., “The cerebrospinal fluid production rate is reduced in dementia of the Alzheimer's type,” Neurology 57: 1763-1766, 2001). Because of the importance of the CSF and its constituents in proper brain function, these age-related changes in the choroid plexus may have important implications for the central nervous system and its disorders.

It would be beneficial to provide methods for detecting and monitoring these and other deleterious changes in CSF production and constituents to allow treatment before significant central nervous system damage occurs or to prevent further progressive damage.

### BRIEF DESCRIPTION OF THE FIGURES

FIG. 1 is a schematic diagram of one embodiment of a device for treating choroid plexus failure.

FIG. 2 is a schematic diagram of a timing mechanism of the device of FIG. 1.

### DETAILED DESCRIPTION OF THE INVENTION

Various embodiments of the present invention provide methods and devices for diagnosing and treating choroid plexus failure in a mammal such as a human. Also provided are methods and devices for diagnosing and treating central nervous system (CNS) disorders. As used herein, choroid plexus failure refers to either (a) the inability of the choroid plexus to produce cerebrospinal fluid (CSF) or one or more of its constituents in normal quantities, or (b) the inability of the choroid plexus to remove noxious substances or metabolic products from the CSF at a satisfactory rate, or (c) both. Choroid plexus failure can result from age-related changes, disease, or drug administration. For example, commonly prescribed drugs, such as diuretics and cardiac glycosides, are known to reduce CSF production rates.

Choroid plexus failure results in one of two general effects, insufficient supply of necessary materials or insufficient removal of toxic materials. Failure of the choroid plexus to produce sufficient quantities of one or more normal constituents of CSF may result in impaired function or disorders of the CNS or its appendages. Failure of the choroid plexus to produce sufficient quantities of CSF itself results in both inadequate amounts of normal constituents of CSF and insufficient turnover of CSF. Insufficient turnover of CSF may result in accumulation of noxious substances or metabolic products that are flushed out when old CSF exits the CNS via the arachnoid villi and is replaced with new CSF. Failure of the choroid plexus to actively remove noxious substances or metabolic products also results in buildup of these products or of products with which they are in equilibrium or to which they are converted. As used herein, CSF components include three different types of non-cellular components: normal constituents of CSF, i.e., those made by the choroid plexus or transported actively by the choroid plexus from the blood to the CSF; noxious substances; and metabolic products.

Because failure of the choroid plexus may result in impairment or disorders of the CNS or its appendages, it is beneficial to detect and treat choroid plexus failure early, in some cases before symptoms of the CNS disorder appear. Typical CNS disorders correlated with choroid plexus failure include cognitive disorders, movement disorders, and sensory disorders.

Diagnosing Choroid Plexus Failure

In one embodiment, the present invention provides a method for diagnosing choroid plexus failure in a subject. A CSF sample is obtained from a subject and levels (e.g., concentrations or absolute amounts) of one or more CSF components, including normal CSF constituents, noxious substances, or metabolic products, is measured. This value or values is compared with a normal value or values to diagnose choroid plexus failure. In general, a measured value that is within about 5%, about 10%, about 15%, or about 20% of the normal value is considered to be normal, i.e., not diagnostic of CSF failure. However, the distance from normal at which choroid plexus failure is diagnosed varies with the particular component. In some cases, only one component is measured, whereas in other cases, a panel of components is measured. The panel of components can include any combination of types of component (normal constituent, noxious substance, or metabolic product).

In another embodiment of the invention, the site or mechanism of choroid plexus failure is diagnosed by measuring the level of one or more CSF components whose level is correlated with a particular choroid plexus action. For example, certain constituents are synthesized by the choroid plexus, others are actively transported from the plasma to the CSF or the CSF to the plasma, and some are pumped into the plasma against a concentration gradient. Measured levels of these particular constituents provide information about whether the failure is in the synthesis of CSF or its constituents, the active transport into or out of the CSF, or the active transport pumping mechanism. Any of these mechanisms can fail independently, or a global failure can include failure of all (or at least two) mechanisms.

In another embodiment of the invention, an individual is selected for treatment by diagnosing choroid plexus failure as described above.

In another embodiment of the invention, choroid plexus failure is diagnosed as described above in a subject taking one or more drugs known to decrease the production of CSF. In this embodiment, drug selection is guided by determining the effect of one or more drugs on choroid plexus function. A drug can then be selected that minimizes its effect on the choroid plexus. Examples of drugs commonly prescribed to the elderly that are known to decrease CSF production include carbonic anhydrase inhibitors, furosemide, amiloride, atrial naturetic factor, cardiac glycosides, cimetidine, ranitidine, omeprazole, vasopressin, some benzodiazepines, and corticosteroids.

A normal value of a level of a CSF component is an approximate known or measured mean value in a healthy population. This value may or may not be described by a normal statistical distribution. As is known in the art, normal values vary depending on characteristics of the described population including, for example, age, sex, geographical location, and ethnicity, as well as on the particular method by which the level is measured. In embodiments of the present invention, the normal values to which measured values are compared may be normal values for an entire population or for a particular sub-population, e.g., males or females, age subgroups, or ethnic groups. It may be beneficial to compare a measured value to a normal value of a population that most closely matches the sub-population to which the subject being diagnosed belongs.

Table 1 is a non-comprehensive list of normal CSF constituents, along with their approximate normal values. These constituents include those secreted by the choroid plexus, those transported from the blood into the CSF by the choroid plexus with or without modification, and those derived from the cerebral interstitial fluid. Embodiments of the invention in which measured values are compared with normal values are not necessarily restricted to the values listed in Table 1. In this embodiment, any CSF component that is currently known or discovered in the future can be measured as a diagnostic of choroid plexus failure.

Any single component or panel of components whose level is indicative of choroid plexus function is considered a biological marker (or biomarker) of choroid plexus function. Biomarkers of choroid plexus function enable early and accurate diagnosis, guide treatment regimens, and aid in the understanding of disease mechanisms. In some cases, the biomarker that is measured is not itself involved in disease mechanisms, but may be correlated with overall CSF production and turnover.

TABLE 1

| Constituent | Concentration |
|---|---|
| Water | 295 mOsm/L |
| Electrolytes | |
| sodium | 138 mEq/L |
| potassium | 2.8 mEq/L |
| calcium | 2.1 mEq/L |
| magnesium | 2.3 mEq/L |
| chloride | 119.0 mEq/L |
| bicarbonate | 22.0 mEq/L |
| CO₂ | 47.0 mm Hg |
| pH | 7.33 |
| O₂ | 43.0 mm Hg |
| Glucose | 60.0 mg/dl |
| Lactate | 1.6 mE/L |
| Pyruvate | 0.08 mEq/L |
| Fructose | 4.0 mg/dl |
| Polyols | 3.4 nmol/dl |
| Myo-inositol | 2.6 mg/dl |
| Total Protein | 35.0 mg/dl |
| prealbumin | 1.4 mg/dl |
| albumin | 22.8 mg/dl |
| alpha-1 globulin | 1.4 mg/dl |
| alpha-2 globulin | 2.8 mg/dl |
| beta globulin | 4.2 mg/dl |
| gamma globulin | 2.5 mg/dl |
| IgG | 1.2 mg/dl |
| IgA | 0.2 mg/dl |
| IgM | 0.06 mg/dl |
| beta-trace protein | 2.0 mg/dl |
| fibronectin | 3.0 mg/dl |
| transferrin | 1.4 mg/dl |
| ceruloplasmin | 0.1 mg/dl |
| amyloid beta-42 | ˜1240 pg/ml |
| Tau | ˜420 pg/ml |
| Amyloid precursor protein | ˜1.6 μl |
| Total free amino acids | 81 mol/L |
| Alanine-citruline | 34.3 μmol/L |
| 2-aminobutyric | 3.5 μmol/L |
| Arginine | 22.4 μmol/L |
| Asparagine | 13.5 μmol/L |
| Aspartate | 150 ng/ml |
| glutamate | 26.1 μmol/L |
| glutamine | 552 μmol/L |
| glycine | 5.9 μmol/L |
| histidine | 12.3 μmol/L |
| isoleucine | 6.2 μmol/L |
| leucine | 14.8 μmol/L |
| lysine | 20.8 μmol/L |
| methionine | 2.5 μmol/L |
| ornithine | 3.8 μmol/L |
| phenylalanine | 9.9 μmol/L |
| phosphoenthanolamine | 5.4 μmol/L |
| phosphoserine | 4.2 μmol/L |
| senne | 29.5 μmol/L |
| taurine | 7.6 μmol/L |
| Threonine | 35.5 μmol/L |
| Tyrosine | 9.5 μmol/L |
| valine | 19.9 μmol/L |
| Other nonprotein nitrogenous | |
| urea | 4.7 nmol/dl |
| creatinine | 1.2 mg/dl |
| uric acid | 0.25 mg/dl |
| putrescine | 184 pmol/ml |
| Spermidine | 150 pmol/ml |
| Total lipids | 1.5 mg/dl |
| free cholesterol | 0.4 mg/dl |
| cholesterol esters | 0.3 mg/dl |
| Mediators (non-hormonal) | |
| cAMP | 20 nmol/L |
| cGMP | 0.68 nmol/L |
| HVA | 35 ng/ml |
| 5-HIAA | 17.1 ng/ml |
| 3 methyoxy-4-hydroxy phenylethylene | 8.1 ng/ml |
| glycol | |
| alpha 1-antichymotrypsin | 0.1 mg/dl |
| Hormones | |
| TRH | 5-40 pg/ml |
| TSH | 0.2-2.65 μU/ml |
| T-4, total | 185 ng/dl |
| free | 5.8 ng/dl |
| T-3, total | 17.4 ng/ml |
| free | 1.8 ng/dl |
| LRH | 11.2 pg/dl |
| LH | 2.4-5.7 mU/ml |
| FSH | 1.7-5.3 mU/ml |
| testosterone | |
| male | 11.1 ng/dl |
| female | 1.4 ng/dl |
| estradiol | |
| male | 3.9 pg/ml |
| female | 2.4 pg/ml |
| progesterone | |
| male | 3.9 ng/dl |
| female | 4.5 ng/dl |
| prolactin | 0.4-1.2 ng/ml |
| somatostatin | 35.4-130 pg/ml |
| growth hormone | 0.35-0.5 ng/ml |
| beta-lipotropin | 89.3 pg/ml |
| beta-endorphin | 6.1 ng/ml |
| methionine-enkephalin | 26.8 ng/ml |
| beta-MSH | 60.1 ng/L |
| ACTH | 27-98 pg/ml |
| cortisol | |
| AM | 0.68 μg/dl |
| PM | 0.38 μg/dl |
| substance p | 1-45 pg/ml |
| arginIne vasopressin | 1.8-2.4 pg/ml |
| calcitonin | 28 pg/ml |
| cholecystokinin | 14 pM |
| gastrin | 3.4 pM |
| VIP | 49.9 pmol/ml |
| insulin | 4-11 μU/ml |
| melatonin | 59 pg/ml |
| bombesin | 34.4 fmol/ml |

Additional normal CSF constituents synthesized or transported by the choroid plexus or derived from the interstitial fluid include, but are not limited to, transthyretin, retinol, retinol-binding protein, ceruloplasmin, prostaglandin, insulin-like growth factors 1 and 2, insulin-like growth factor binding proteins, cystatin-C, leptin, thyroxine, ascorbate, thiamine phosphate, pyridoxine phosphate, methyltetrahydrofolate, alpha 1-antichymotrypsin, alpha 2-macroglobulin, prothrombin, beta 2-microglobulin, proapolipoprotein, apolipoprotein E, ubiquitin, prostaglandin D₂ synthase (β-trace protein), glutamine synthetase, tau, neuron-specific enolase, and S-100B.

CSF components can be measured using conventional methods of obtaining samples and measuring concentrations or other levels. Measurement includes measuring all possible levels, including zero levels or those too low to be detected by the technique used. The measurement may be direct, i.e., requiring contact with the CSF, or indirect, using techniques such as nuclear magnetic resonance (NMR) or radio spectroscopy. Direct measurement can be performed by obtaining CSF from a patient or by placing a sensor within the CSF for temporary or permanent measurement of one or more components.

CSF sampling can occur by any known means, including, but not limited to, spinal tap (lumbar puncture), cisternal puncture, ventricular sampling, or implanted access port (see, e.g., U.S. Pat. No. 6,383,159, issued to Saul et al., and U.S. Pat. No. 5,897,528, issued to Schultz, both of which are incorporated herein by reference, as are the references therein). In some cases, CSF may be sampled from a leak due to injury (e.g., to the cribriform plate with leakage through the nostrils). A single sample may be acquired, or multiple samples may be acquired continuously or at various times to monitor therapeutic effects or other changes.

Concentrations or other levels of CSF components can be measured by any technique known in the art, including, for example, spectrophotometric, fluorometric, and chemiluminescent methods, ELISA, immunoassay, mass spectrometry, and x-ray fluorescence. In some cases, a metabolite of a component or a surrogate marker may be measured instead of, or in addition to, the component itself. Additionally, when levels are measured by placing a sensor within the body, a variety of means are available for transmitting the signal of the embedded sensor, e.g., by telemetry.

In some cases, it may be possible to measure CSF components in the blood or urine, which are much easier to obtain from a patient than is CSF. Normal levels of components in the blood and urine differ from those in the CSF and must be determined before diagnosis can occur.

A variety of disorders of the CNS or its appendages may result from insufficient or overabundant levels (e.g., about 5%, about 10%, about 15%, or about 20% above or below normal) of any normal CSF constituents, such as those listed above or in Table 1. In addition, CSF has been shown by two-dimensional gel electrophoresis to contain at least 480 protein constituents, many of which have not been identified but may play a role in normal or pathological CNS processes, and may be measured in embodiments of the present invention. In one embodiment of the invention, a CNS disorder is diagnosed by diagnosing choroid plexus failure as described above. Examples of diseases or disorders that can be diagnosed include cognitive disorders, such as impaired memory, aphasia, apraxia, agnosia, impaired executive, social, or occupational functioning, and fully evolved dementia; movement disorders, such as motor weakness or paralysis, loss of coordination, abnormalities of posture, stance, gait, or locomotion, spasm, tics, myoclonus, tremor, athetosis, chorea, and dystonia; sensory disorders such as disorders of vision, hearing, taste, sense of smell, proprioception, touch, position, and pain abnormalities; and speech disorders. These disorders may be genetic, congenital, or acquired.

For example, a number of CNS disorders are caused by a below-normal level of transthyretin in the CSF. Transthyretin is produced in large quantities by the choroid plexus and accounts for up to 50% of all proteins secreted by the choroid plexus; almost all of the transthyretin in the CSF is produced by the choroid plexus. Transthyretin is a carrier protein that transports the thyroid hormone thyroxine into the brain by crossing the ventricular ependyma to enter the periventricular cortex, thalamus, hypothalamus, and basal ganglia. Without transthyretin, thyroxine can reach cortical regions of the brain via receptors on the cerebral capillary endothelium. Below-normal levels of transthyretin in the CSF may lead to hypothyroidism in the periventricular regions of the brain, causing psychomotor retardation, depression, and cognitive decline in the absence of peripheral manifestations of hypothyroidism. In fact, low levels of transthyretin have been found in the CSF of patients with depression and with dementia of the Alzheimer's type. See, e.g., M. B. Dratman et al., “Transport of iodothyronines from bloodstream to brain: contributions by blood:brain and choroid plexus:cerebrospinal fluid barriers,” Brain Res 554: 229-236, 1996; G. M Sullivan et al., “Low levels of transthyretin in the CSF of depressed patients,” Am J Psychiatry 156: 710-715, 1999; J.-M. Serot et al., “Cerebrospinal fluid transthyretin: aging and late onset Alzheimer's,” J Neurol Neurosurg Psychiatry 63: 506-508, 1997; A. Merched et al., “Apolipoprotein E, transthyretin and actin in the CSF of Alzheimer's patients: relation with the senile plaques and cytoskeleton biochemistry,” FEBS Lett 425: 225-228, 1998; H. Riisoen, “Reduced prealbumin (transthyretin) in CSF of severely demented patients with Alzheimer's disease,” Acta Neurol Scand 78: 455-459, 1988; and M. J. Saraiva, “Transthyretin mutations in hyperthyroxinemia and amyloid diseases,” Hum Mutat 17: 493-503, 2001, all of which are incorporated herein by reference. In embodiments of the present invention, choroid plexus failure or diencephalic hypothyroidism are diagnosed by measuring transthyretin or thyroxine levels in the CSF.

Transthyretin also ferries retinol-binding protein, receptors for which are found in regions of the cerebral capillary endothelium as well as in the epithelium of the choroid plexus. A below-normal level of transthyretin in the CSF, therefore, may lead to disorders associated with a deficiency in retinol. Retinoids are known to be essential for the development of the CNS, although their role is not fully elucidated in the adult brain. Retinoic acid is present throughout the brain and spinal cord of the adult and has been shown to be a regulatory transcription factor for the expression of dopamine D2R receptors in the adult mouse brain and pituitary gland. Mice lacking these dopamine receptors develop a locomotor disorder similar to that seen in Parkinsonism. Additionally, brain regions of patients with Alzheimer's disease show higher activity of retinaldehyde dehydrogenase, the enzyme that converts retinaldehyde to retinoic acid, than do control brains, indicating a deficiency in retinol. This increased enzyme activity disappears when retinol is added to the brain tissue. See, e.g., J. A. Hamilton et al., “Transthyretin: a review from a structural perspective,” Cell Mol Life Sci 58: 1491-1521, 2001; P. N. MacDonald et al., “Localization of Cellular Retinol-Binding Protein and Retinol-Binding Protein in Cells Comprising the Blood-Brain Barrier of Rat and Human,” Proc Natl Acad Sci USA 87: 4265-4269, 1990; A. Gavalas, “ArRAnging the hindbrain,” Trends Neurosci 25: 61-64, 2002; E. A. Werner and H. F. Deluca, “Retinoic acid is detected at relatively high levels in the CNS of adult rats,” Am J Physiol Endocrinol Metab 282: E672-E678, 2002; G. Wolf, “Vitamin A functions in the regulation of the dopaminergic system in the brain and pituitary gland,” Nutr Rev 56: 354-358, 1998; and M. J. Connor and N. Sidell, “Retinoic acid synthesis in normal and Alzheimer diseased brain and human neural cells,” Mol Chem Neuropathol 30: 239-252, 1997, all of which are incorporated herein by reference.

The choroid plexus also secretes prostaglandin D₂ synthase, the enzyme responsible for synthesis of prostaglandin D₂, which regulates sleep in mammals. Prostaglandin D₂ is the principal prostanoid in the mammalian CNS. Insufficient production of prostaglandin D₂ synthase by the choroid plexus may result in sleep disorders. For more information, see, e.g., O. Hayaishi, “Prostaglandin D₂ and sleep—a molecular genetic approach,” J Sleep Res 8: 60-64, 1999; and Y. Urade et al., “Dominant Expression of mRNA for Prostaglandin D Synthase in Leptomeninges, Choroid Plexus, and Oligodrendrocytes of the Adult Rat Brain,” Proc Natl Acad Sci USA 90: 9070-9074, 1993, both of which are incorporated herein by reference.

The choroid plexus supplies the brain with a number of micronutrients, including ascorbate (vitamin C), thiamine (vitamin B₁), pyridoxine (vitamin B₆), and folates. In some cases, the micronutrients are modified in the choroid plexus before being released into the CSF. For example, the nonphosphorylated forms of thiamine and pyridoxine circulate in the plasma and accumulate in the choroid plexus epithelial cells before being phosphorylated and released in the CSF as monophosphates. Abnormally low or high levels of these micronutrients can cause a variety of disorders. See, e.g., R. Spector, “Micronutrient homeostasis in mammalian brain and cerebrospinal fluid,” J Neurochem 53: 1667-1674, 1989, which is incorporated herein by reference.

In addition to deficiencies in production of CSF or its constituents, failure in the active transport mechanism of the choroid plexus may have deleterious effects. For example, methyltetrahydrofolate is actively transported from the plasma to the CSF, where it has a concentration four times that in the plasma. Although the effect of folate deficiency in the CSF is not fully known, a variety of effects have been observed. For example, folate deficiency predisposes patients to hyperhomocysteinemia, a significant risk factor for dementia and Alzheimer's disease in humans. In mice, folic acid deficiency and high homocysteine levels have been shown to lead to DNA damage and increase the toxicity to neurons of amyloid beta (A-beta) peptide. Mice bearing a mutation involving amyloid precursor protein (APP) suffered increased DNA damage and hippocampal neurodegeneration as a result of a folate-deficient diet. One study of 1077 people aged 60 to 90 who underwent brain MRI found silent brain infarcts and white matter lesions to be strongly associated with total plasma homocysteine levels, again implicating folate deficiency. CSF folate levels have been found to be reduced in patients with late-onset dementia of the Alzheimer's type. In one study, an adult male of Dutch ancestry was found to have a low level of folate in the CSF and normal levels in the plasma and red blood cells, presumably owing to a defect in active transport by the choroid plexus. His neurologic abnormalities included cerebellar dysfunction, distal spinal muscular atrophy, pyramidal tract abnormalities, and sensory hearing loss. For more information on folate deficiency, see, e.g., R. A. Wevers et al., “Folate deficiency in cerebrospinal fluid associated with a defect in folate binding protein in the central nervous system,” J Neurol Neurosurg Psychiatry 57: 223-226, 1994; S. Seshadri et al., “Plasma homocysteine as a risk factor for dementia and Alzheimer's disease,” N Engl J Med 346: 476-483, 2002; I. I. Kruman et al., “Folic acid deficiency and homocysteine impair DNA repair in hippocampal neurons and sensitize them to amyloid toxicity in experimental models of Alzheimer's disease,” J Neurosci 22: 1752-1762, 2002; S. E. Vermeer et al., “Homocysteine, silent brain infarcts, and white matter lesions: The Rotterdam Scan Study,” Ann Neurol 51: 285-289, 2002; and J.-M. Serot et al., “CSF-folate levels are decreased in late-onset AD patients,” J Neural Transm 108: 93-99, 2001, all of which are incorporated herein by reference.

Other CSF constituents that are transported actively from the plasma by the choroid plexus include vitamin B₁₂, vitamin E, and ascorbate (vitamin C). The concentration of vitamin C is four times greater in the CSF than in the plasma, and transport across the blood-brain barrier is minimal. Failure by the CSF to actively transport these substances may result in a variety of disorders.

CNS disorders can also result from accumulation of noxious substances, including: (1) normal CSF constituents that are not harmful (or even essential) at normal levels but are toxic at elevated levels, e.g., metals such as copper, zinc, or iron; (2) normal waste products that accumulate to toxic levels; and (3) harmful compounds that are absent or at negligible levels in healthy individuals. The level of noxious substances at which choroid plexus failure can be diagnosed varies widely with the particular noxious substance. Noxious substances may be produced during, result in, or characterize pathological states.

Normal waste products can accumulate if the CSF production rate drops below normal or if active transport mechanisms fail. In young, healthy humans, the production rate of CSF is about 0.4-0.6 ml/min, equivalent to a daily production of approximately 700 ml of CSF. For an average total CSF volume of approximately 150-175 ml, the daily turnover rate is roughly four times. As this rate drops, the concentration of waste products can increase above normal levels. Active transport removes waste products, drugs, metabolic derivatives of neurotransmitters, and mediators of inflammation such as leukotrienes. Examples of waste products that may accumulate to toxic levels as a result of choroid plexus failure include ammonia, glutamine, and certain amino acids.

Harmful compounds that are not normal waste products may also accumulate as a result of choroid plexus failure. Examples of harmful compounds include exogenous agents such as iodine and penicillin, homocysteine, and A-beta peptide, which is the precursor of insoluble amyloid, deposits of which arc found in patients with Alzheimer's disease. Alzheimer's disease is a genetic disorder transmitted by autosomal dominance and characterized by one of three possible mutations, either in amyloid precursor protein (APP) or in presenilin 1 or 2. The presenilins cleave APP to release the A-beta peptide, which auto-assembles into amyloid. In the present invention, reduced CSF turnover leads to reduced clearance of the A-beta peptide, shifting the equilibrium between peptide and precipitate toward deposition of insoluble amyloid, resulting in neuronal damage and death. Experiments in rats have shown that A-beta peptide is normally cleared from the ventricular CSF into the blood within ten minutes of intraventricular injection. The choroid plexus has specific receptors for A-beta peptide, indicating that it also actively transports the peptide from the CSF, and that choroid plexus failure may hamper this active transport mechanism. Evidence for the effect of shifting the A-beta/amyloid equilibrium is found in Down syndrome, a disorder characterized by a chromosomal abnormality, specifically a trisomy of chromosome 21, which contains the gene for APP. Down syndrome patients exhibit premature aging with an Alzheimer's-like disease having amyloid deposits, caused presumably by the overproduction of APP and A-beta peptide and the shift of the equilibrium toward amyloid deposition. In the case of choroid plexus failure, the problem is not overproduction of APP, but rather failure to clear this soluble precursor of amyloid.

In one embodiment of the invention, insufficient CSF production is diagnosed by measuring levels of one or more CSF constituents. In some cases, these constituents are derived from the interstitial fluid and not produced by the choroid plexus, but buildup in the levels is indicative of insufficient CSF production or insufficient active transport by the choroid plexus. Generally, buildup of one or more noxious substances or waste products can be inferred from knowledge about CSF production, which can itself be determined by measuring the levels of an unrelated CSF component. That is, the component that is measured as an indication of choroid plexus failure may itself have no known deleterious effects when its level is above or below normal.

Treating Choroid Plexus Failure

An alternative embodiment of the present invention is a method for treating choroid plexus failure in a subject by administering to the subject one or more normal CSF constituents, including any of those listed above and in Table 1. Alternatively, a CSF sample is obtained and levels of one or more CSF components measured (as described above) and the level adjusted toward a desired level (e.g., normal level), either by adding a normal CSF constituent, by administering a pharmacological agent, or by removing a noxious agent or metabolic product. Note that treatment can occur without previous measurement of the level of one or more CSF components. Note also that the desired level to which the component is adjusted may be a level other than (above or below) the normal level.

As used herein, treating (or treatment) is an approach for obtaining beneficial or desired results, including clinical results. These results include, without limitation, at least one of alleviating symptoms; diminishing or stabilizing the extent or progression of the disease or disorder; preventing the spread, occurrence, or reoccurrence of the disease or disorder; and reducing the probability of occurrence of the disease or disorder or its symptoms. Treatment may result in reduction of pathological consequences such as amyloid deposition.

In these embodiments, an effective amount of the CSF constituent can be administered to result in a desired level such as the normal level or a value within about 5%, about 10%, about 15%, or about 20% of the desired level. Alternatively, a pharmacological agent is administered to the subject that results in a desired change in the level of one or more CSF components. The pharmacological agent can be, for example, a drug that causes a change in the concentration of a CSF constituent toward a desired level, a precursor of a compound that is a normal CSF constituent, or a pharmacological agent that acts directly on the cells or vasculature of the choroid plexus. More than one pharmacological agent or CNS constituent, or combinations of each, may be administered. The measurement, comparison (of measured and desired levels), and administration steps can occur periodically, continuously, sequentially, or simultaneously.

One example of a drug administered to adjust the level of a CSF component is nicotine, which increases the production of transthyretin by the choroid plexus. Cigarette smoking has been shown to improve age-related dementia and certain forms of depression. See, e.g., M. D. Li et al., “Nicotine enhances the biosynthesis and secretion of transthyretin from the choroid plexus in rats: implications for beta-amyloid formation,” J Neurosci 20: 1318-1323, 2000; and E. D. Levin and A. H. Rezvani, “Development of nicotinic drug therapy for cognitive disorders,” Eur J Pharmacol 393: 141-146, 2000, both of which are incorporated herein by reference.

Other drugs can decrease the production rate of CSF. Examples include, but are not limited to, carbonic anhydrase inhibitors, furosemide, amiloride, atrial naturetic factor, cardiac glycosides, cimetidine, ranitidine, omeprazole, vasopressin, some benzodiazepines, and corticosteroids. See, e.g., H. Davson and M. B. Segal, eds., Physiology of the CSF and blood-brain barriers. Boca Raton: CRC Press, pp. 214-217, 1996; and F. M. Faraci et al., “Effect of endogenous vasopressin on blood flow to choroid plexus during hypoxia and intracranial hypertension,” Am J Physiol 266: H393-H398, 1994, both of which are incorporated herein by reference. Pharmacological agents that influence the central nervous system or its blood supply include drugs used in treating Alzheimer's disease, such as cholinesterase inhibitors (S. Gauthier, “Advances in the pharmacotherapy of Alzheimer's disease,” CMAF 166: 616-623, 2002, which is incorporated herein by reference).

Precursors of normal CSF constituents that are secreted by the choroid plexus include the vitamers thiamine and pyridoxine, which are phosphorylated in the choroid plexus cells and secreted into the CSF in their phosphorylated form. These vitamers can be administered in embodiments of the present invention to increase levels of the phosphorylated forms in the CSF.

In another embodiment of the present invention, the level of one or more noxious substances in the CSF is lowered, e.g., by dialysis or by removing a portion of the CSF from the subarachnoid space and transporting it via an implanted path (shunting it) to another part of the patient's body.

In one embodiment of the invention, a disorder of the CNS or its appendages is treated by treating choroid plexus failure as described above. Examples of diseases or disorders that can be treated include cognitive disorders, such as impaired memory, aphasia, apraxia, agnosia, impaired executive, social, or occupational functioning, and fully evolved dementia; movement disorders, such as motor weakness or paralysis, loss of coordination, abnormalities of posture, stance, gait, or locomotion, spasm, tics, myoclonus, tremor, athetosis, chorea, and dystonia; sensory disorders such as disorders of vision, hearing, taste, sense of smell, proprioception, touch, position, and pain abnormalities; and speech disorders. These disorders may be genetic, congenital, or acquired. Symptoms of the disorders may vary in type and extent, and may be detected before symptom appearance by techniques such as MRI scans, laboratory tests, or other suitable methods.

The CSF constituent or constituents or pharmacological agent can be administered in a single dose, in a series of single doses, in a sustained release form, in a non-sustained release form, continuously over time at a controlled rate, or according to any other suitable administration regimen. If different compounds are administered, each can be administered in a different form by a different route. Combination administration methods include, for example, an initial dose in both non-sustained release form and sustained release form; an initial bolus followed by continuous infusion; one compound administered in sustained release form and another in a form allowing faster or slower release; and a pharmacological agent administered in conjunction with one or more normal CSF constituents.

The compound or compounds can be administered by any route that results in entry into the CSF or an effect on the CSF or on the choroid plexus. Note that if the compound is a pharmacological agent that acts directly on the choroid plexus, it can enter the choroid plexus via the plasma and does not need to be transported into the CSF. Suitable administration routes include, for example, intrathecally (into the subarachnoid space), e.g., by lumbar puncture or via an implanted access port; systemically (e.g., parenterally, gastrointestinally, or via naso-pharyngeal and pulmonary routes); or by any combination of routes. If a CSF constituent is administered by a route that is not an intrathecal route, the constituent must be able to gain access to the CSF. Some CSF constituents or precursors (e.g., vitamers) may be provided systemically (e.g., orally or intravenously) and can increase the blood concentration sufficiently to drive entry of the compound or its modified form into the CSF, either across the blood-brain barrier or via transport by the choroid plexus. Some drugs can cross the blood-brain barrier only after brain damage has occurred.

Sustained release forms of compounds can be formulated by any means known in the art. Currently, only normal constituents of the CSF can be administered intrathecally, and so a sustained release formulation for intrathecal administration may include a compound or pharmacological agent encapsulated in a dried form (e.g., in a wafer) of a normal constituent of CSF such as glucose, amino acids, or albumin. Future sustained release formulations may be able to be administered intrathecally without normal CSF constituents, and all such formulations are within the scope of the present invention. For substances that are not administered intrathecally, other sustained-release formulations, such as dispersions in polymeric matrices or encapsulation in polymer layers, may be employed. Methods and compositions for sustained release of compounds into the subarachnoid space are described in the following patents, all incorporated herein by reference: U.S. Pat. No. 6,187,906, issued to Gluckman et al.; U.S. Pat. No. 6,123,956, issued to Baker et al.; and U.S. Pat. No. 5,576,018, issued to Kim et al.

When administered by a non-intrathecal route, the compound can be combined with a pharmaceutically acceptable excipient, a relatively inert substance that facilitates administration of the compound, e.g., by giving form or consistency to the composition or acting as a diluent. Suitable excipients include, but are not limited to, stabilizing agents, wetting and emulsifying agents, salts for varying osmolarity, encapsulating agents, buffers, and skin penetration enhancers.

Continuous administration of a compound at a controlled rate can be achieved by dialysis using a semipermeable membrane and an access port to the CSF (see, e.g., U.S. Pat. No, 5,980,480, issued to Rubenstein et al., which is incorporated herein by reference). This method may also be used to remove noxious agents from the CSF. Other methods of continuous infusion include an implanted reservoir (e.g., an Ommaya-type reservoir), in which the constituent or pharmacological agent is contained in encapsulated or other form allowing sustained release, or a reservoir incorporated into a stand-alone infusing system or a CSF shunt.

Dosages, administration rates, and removal rates can be estimated or calculated based on a comparison between the measured level of a CSF component and a normal or other desired level of the component. Other relevant factors include the turnover rate and volume of CSF, which can be determined by known methods (see, e.g., A. C. Mamourian et al., “Visualization of Intravenously Administered Contrast Material in the CSF on Fluid-Attenuated Inversion-Recovery MR Images: An In Vitro and Animal-Model Investigation,” Am J Neuroradiol 21: 105-111, 2000; A. Bozzao et al., “Cerebrospinal fluid changes after intravenous injection of gadolinium chelate: assessment by FLAIR MR imaging,” Eur Radiol 13: 592-597, 2003; J. Masserman, “Cerebrospinal fluid hydrodynamics. IV. Clinical experimental studies,” Arch Neurol Psychiatry 32: 524-553, 1934; and P. Gideon et al., “Cerebrospinal fluid production and dynamics in normal aging: a MRI phase-mapping study,” Acta Neurol Scand 89: 362-366, 1994, all of which are incorporated herein by reference). A dosage or administration rate can be computed that will achieve a level of the component that is within about 5%, about 10%, about 15%, about 20%, about 25%, or about 50% of the normal value listed in Table 1 or other desired value. When CSF is shunted to another area of the body, the flowrate may be calculated by any suitable means, such as those provided in the following patents, all incorporated herein by reference: U.S. Pat. No. 5,980,480, issued to Rubenstein et al.; U.S. Pat. No. 6,264,625, issued to Rubenstein et al.; U.S. Pat. No. 5,385,541, issued to Kirsch et al.; and U.S. Pat. No. 4,950,232, issued to Ruzicka et al. The selected flowrate is not greater than the rate of production of CSF by the choroid plexus, e.g., between about 17 and about 25 mL/hour.

In one embodiment, the level of one or more CSF constituents is monitored intermittently or continuously and the rate or form of administration adjusted accordingly. Adjustments may be computed manually or automatically based on standard algorithms known in the art.

Therapeutic Device

One embodiment of the present invention is a device for treating choroid plexus failure in an animal such as a vertebrate or mammal, e.g., a human. Alternatively, the device is used to treat a disorder of the CNS or its appendages. The device can be configured to perform one or more of the following functions: measuring the level of one or more CSF components, administering CSF constituents or pharmacological agents to the subarachnoid space or other region, and shunting CSF from the subarachnoid space to another region of the body. In some embodiments, the device is implantable.

Generally, the device includes a reservoir and means for delivering material in the reservoir to the subarachnoid space of a subject. The device can also contain means for regulating the delivery rate of material to the subarachnoid space.

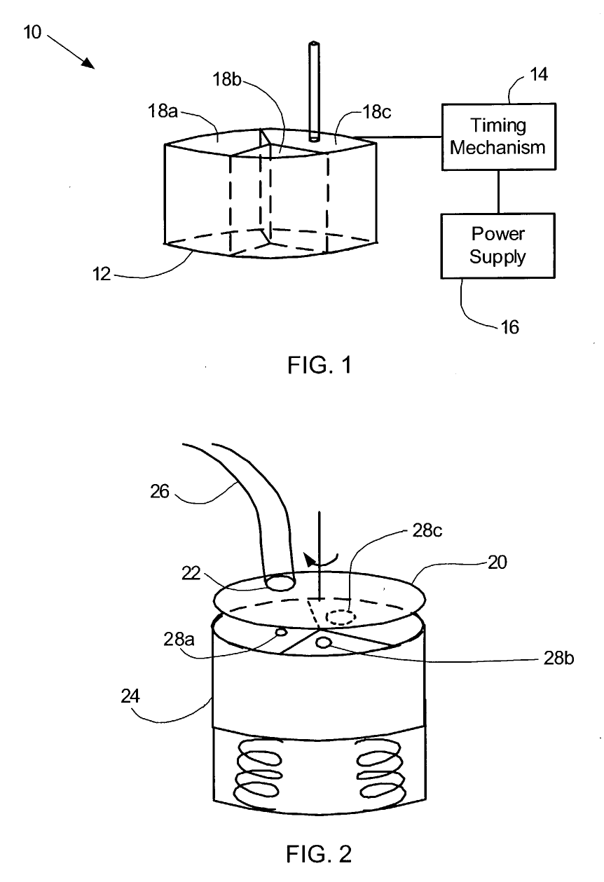

FIG. 1 illustrates schematically one embodiment of the invention, an implantable device 10 for controlled release of one or more repletion agents (normal CSF constituents) into the CSF for treating choroid plexus failure. The device can be implanted in the cranium or subcutaneously, e.g., in the infraclavicular fossa (below the collarbone), the abdominal wall, or the lumbar region. The implanted device includes a reservoir 12 containing the material to be delivered, an electronic timing mechanism 14 for regulating the flowrate of material into the CSF, and a power supply 16, such as one or more long-life miniature batteries. The entire device 10, the reservoir 12, the repletion agents, or the battery 16 can be replaced by minimally invasive access to the subcutaneous device.

In one embodiment, the reservoir 12 is subdivided into separate compartments 18a-18c. The total reservoir capacity is between about 10 ml and about 100 ml, or between about 30 ml and about 60 ml, with specific values within these ranges selected based upon the particular administration regimen. The total capacity required depends upon the difference between the normal and measured levels of the CSF component, the CSF volume and turnover rate, and the solubility of the substance, among other factors. Repletion agents that are chemically compatible with each other can be stored in a single reservoir compartment, whereas substances that are incompatible with each other or require widely varying dilution levels to maintain solubility are stored in separate compartments. If necessary, substances can be chemically isolated during delivery by providing a bolus of isotonic saline, stored in one or more intervening compartments, between substances.

In one embodiment, the timing mechanism 14 consists of a vibrating quartz crystal supplied by the power supply 16, an integrated circuit controlling the oscillation of the quartz crystal, a stepper motor controlled by the integrated circuit, a trimmer element that regulates frequency, a gear train, and a time-regulated component that regulates the delivery rate of the repletion agents. For example, as shown in FIG. 2, the time-regulated component can be a rotating disc 20 containing one or more apertures 22 connecting a reservoir 24 to an element that directs the material to the subarachnoid space (e.g., a catheter 26). Each compartment of the reservoir 24 is sealed by a top surface that has an aperture 28a-28c of varying diameter. As the disc 20 rotates under the control of the timing mechanism, it comes into registration with each aperture 28. The reservoir 24 containing the material is pressurized (e.g., spring-loaded) to urge the material through the apertures 22 and 28 into the catheter 26 and toward the subarachnoid space. The rate of rotation of the disc 20, the pressure on the material, and the area of the aperture 28 determine the delivery rate of material. As will be appreciated by one of ordinary skill in the art, many mechanisms can be used to control the delivery rate of material, and any suitable structure can be employed in embodiments of the present invention.

In one embodiment, a single catheter is provided whose terminal end is introduced into a (non-dominant) lateral ventricle of the subject. Alternatively, two catheters can be used, one inserted into the lateral ventricle and the other inserted across the septum pellucidum into the opposite lateral ventricle, or each introduced independently into separate lateral ventricles. The two-catheter configuration may be necessary if there is inadequate mixing of the repletion agents into the two ventricles. Multiple holes in each catheter increase the mixing of repletion agents within the CSF.

When the device is first implanted, it is necessary to fill the catheter with either saline or one or more repletion agents to prevent delivery of air into the ventricles.

In an alternative embodiment, the device includes an apparatus for removing CSF, which includes a conduit with first and second openings, the first in fluid communication with a subject's subarachnoid space and the second in fluid communication with a different part of the subject's body; and a flowrate control device attached to the conduit between first and second openings, containing a spring-loaded axially reciprocatable cylinder inside a tubular lumen, such that fluid flow through the lumen causes the cylinder to further enter the lumen to increase flow resistance.

In another embodiment of the invention, when choroid plexus failure occurs in one lateral ventricle only, choroid plexus tissue is transplanted from one lateral ventricle to the other. After transplantation, the choroid plexus tissue continues to perform its normal functions of producing CSF and its constituents and actively transporting material between the blood and CSF.

It should be noted that the foregoing description is only illustrative of the invention. Various alternatives and modifications can be devised by those skilled in the art without departing from the invention. Accordingly, the present invention is intended to embrace all such alternatives, modifications and variances which fall within the scope of the disclosed invention.

What is claimed is:

1. A method for diagnosing choroid plexus failure in a subject, comprising:
a) measuring a level of at least one component in the cerebrospinal fluid (CSF) of said subject; and
b) comparing said measured level to a normal level to diagnose choroid plexus failure.

2. The method of claim 1, further comprising obtaining a CSF sample from said subject.

3. The method of claim 1, wherein said component is a normal constituent of CSF.

4. The method of claim 3, wherein said normal constituent is at least one of transthyretin, transferrin, ceruloplasmin, prostaglandin, D-synthase, cystatin-C, leptin, insulin-like growth factor (IGF)-1, IGF-2, IGF binding proteins, retinol, retinol-binding protein, thyroxine, ascorbate, thiamine, thiamine phosphate, pyridoxine, pyridoxine phosphate, proapolipoprotein, apolipoprotein E, ubiquitin, prostaglandin D₂ synthase, tau, neuron-specific enolase, S-100B, and methyltetrahydrofolate.

5. The method of claim 1, wherein said component is a noxious substance.

6. The method of claim 5, wherein said noxious substance is amyloid beta peptide.

7. The method of claim 1, wherein said component is a metabolic product.

8. A method for diagnosing a central nervous system disorder, comprising diagnosing choroid plexus failure according to the method of claim 1.

9. The method of claim 8, wherein said disorder is at least one of a cognitive disorder, a movement disorder, a sensory disorder, dementia, diencephalic hypothyroidism, psychomotor retardation, depression, and folic acid deficiency.

10. A method for treating choroid plexus failure in a subject, comprising administering to said subject at least one constituent of cerebrospinal fluid (CSF).

11. The method of claim 10, wherein said at least one constituent is at least one of transthyretin, transferrin, ceruloplasmin, prostaglandin, D-synthase, cystatin-C, leptin, insulin-like growth factor (IGF)-1, IGF-2, IGF binding proteins, retinol, retinol-binding protein, thyroxine, ascorbate, thiamine, thiamine phosphate, pyridoxine, pyridoxine phosphate, proapolipoprotein, apolipoprotein E, ubiquitin, prostaglandin D₂ synthase, tau, S-100B, and methyltetrahydrofolate.

12. The method of claim 10, further comprising implanting a reservoir containing said constituent in said subject.

13. The method of claim 10, further comprising measuring a level of said constituent in the cerebrospinal fluid of said subject.

14. The method of claim 10, wherein said constituent is administered continuously at a controlled rate.

15. The method of claim 10, wherein said constituent is administered into the subarachnoid space of said subject.

16. A method for treating a central nervous system disorder in a subject, comprising treating choroid plexus failure according to the method of claim 10.

17. The method of claim 16, wherein said disorder is at least one of a cognitive disorder, a movement disorder, a sensory disorder, dementia, diencephalic hypothyroidism, psychomotor retardation, depression, and folic acid deficiency.

18. A method for treating choroid plexus failure in a subject, comprising:
a) measuring a level of at least one component in the cerebrospinal fluid (CSF) of said subject; and
b) based on said measurement, adjusting the level of said component in said CSF.

19. The method of claim 18, wherein said component is a CSF constituent and adjusting the level of said component comprises administering said CSF constituent to said subject.

20. The method of claim 18, wherein adjusting the level of said component comprises administering a pharmacological agent to said subject.

21. The method of claim 18, wherein said component is a noxious substance and adjusting the level of said component comprises removing said noxious substance from the cerebrospinal fluid of said subject.

22. The method of claim 21, wherein said noxious substance is amyloid beta peptide.

23. An implantable device for treating choroid plexus failure in a subject, comprising:
a) a reservoir; and
b) means for delivering material in said reservoir to the subarachnoid space of said subject.

24. The device of claim 23, further comprising means for regulating the delivery rate of material to the subarachnoid space of said subject.

25. The device of claim 23, further comprising at least one catheter having a terminal end adapted for insertion into a lateral ventricle of said subject.

26. The device of claim 23, wherein said reservoir comprises at least two compartments.

27. The device of claim 23, wherein said reservoir has a volume of between about 10 ml and about 100 ml.

28. The device of claim 23, wherein said reservoir has a volume of between about 30 ml and about 60 ml.
